(19) United States
(12) Patent Application Publication
(10) Pub. No.: US 2010/0010034 A1
(43) Pub. Date: Jan. 14, 2010

(54) CRTH2 ANTAGONISTS

(76) Inventors: George Hynd, Essex (GB); Nicholas Charles Ray, Essex (GB); Harry Finch, Essex (GB); David Middlemiss, Essex (GB); Michael Colin Cramp, Essex (GB); Paul Matthew Blaney, Essex (GB); Karen Williams, Essex (GB); Yann Griffon, Essex (GB); Trevor Keith Harrison, Essex (GB); Peter Crackett, Essex (GB)

Correspondence Address: SALIWANCHIK LLOYD & SALIWANCHIK;A PROFESSIONAL ASSOCIATION, PO Box 142950, GAINESVILLE, FL, 32614, US

(21) Appl. No.: 12/519,979

(22) PCT Filed: Dec. 21, 2006
(86) PCT No.: PCT/GB06/04856

§ 371 Date: Aug. 5, 2009

(51) Int. Cl. A61K 31/44; C07D 471/04; A61P 11/00; A61P 1/00

(52) U.S. Cl. 514/299; 546/112

(57) ABSTRACT

The following compounds are CRTH2 antagonists, useful in treatment of respiratory disease: [3-(2,4-dichlorophenylsulfanyl)-6-fluoro-2-methylindolizin-1-yl]acetic acid, [6-fluoro-3-(2-fluoro-4-methanesulfonylphenylsulfanyl)-2-methylindolizin-1-yl]acetic acid, [6-fluoro-3-(4-methanesulfonyl-2-trifluoromethylphenylsulfanyl)-2-methylindolizin-1-yl]acetic acid, (R)-2-[6-fluoro-3-(4-methanesulfonylphenylsulfanyl)-2-methylindolizin-1-yl]propionic acid, [3-(4-ethanesulfonylphenylsulfanyl)-6-fluoro-2-methylindolizin-1-yl]acetic acid, (S)-2-[6-fluoro-3-(4-methanesulfonylphenylsulfanyl)-2-methylindolizin-1-yl]propionic acid, ethanesulfonylaminobenzenesulfonyl)-6-fluoro-2-methyiindolizin-1-yl]acetic acid, [7-chloro-6-fluoro-3-(4-methanesulfonylphenylsulfanyl)-2-methylindolizin-1-yl]acetic acid, [3-(2-chloro-4-methanesulfonylphenylsulfanyl)-7-cyano-2-methylindolizin-1-yl]acetic acid, [6-cyano-3-(4-methanesulfonylbenzyl)-2-methylindolizin-1-yl]acetic acid, [3-(4-chlorobenzyl)-7-cyano-2-methylindolizin-1-yl]acetic acid, [6-cyano-3-(6-fluoroquinolin-2-yl-methyl)-2-methylindolizin-1-yl]acetic acid, [6-fluoro-3-(4-methoxyphenylsulfanyl)-2-methylindolizin-1-yl]acetic acid, [7-chloro-6-fluoro-3-(4-methoxyphenylsulfanyl)-2-methylindolizin-1-yl]acetic acid, [3-(4-bromophenylsulfanyl)-6-fluoro-2-methylindolizin-1-yl]acetic acid, and [3-(4-cyclopropylsulfamoylphenylsulfanyl)-6-fluoro-2-methylindolizin-1-yl]acetic acid.

This invention relates to specific indolizine compounds which are ligands of the CRTH2 receptor (Chemoattractant Receptor-homologous molecule expressed on T Helper cells type 2), and their use in the treatment of diseases responsive to modulation of CRTH2 receptor activity, principally diseases having a significant inflammatory component.

### BACKGROUND TO THE INVENTION

Mast cells are known to play an important role in allergic and immune responses through the release of a number of mediators, such as histamine, leukotrienes, cytokines, prostaglandin D₂, etc (Boyce; Allergy Asthma Proc., 2004, 25, 27-30). Prostaglandin D₂ (PGD₂) is the major metabolite produced by the action of cyclooxygenase on arachadonic acid by mast cells in response to allergen challenge (Lewis et al; J. Immunol., 1982, 129, 1627-1631). It has been shown that PGD₂ production is increased in patients with systemic mastocytosis (Roberts; N. Engl. J. Med., 1980, 303, 1400-1404), allergic rhinitis (Naclerio et al; Am. Rev. Respir. Dis., 1983, 128, 597-602; Brown et al; Arch. Otolarynol. Head Neck Surg., 1987, 113, 179-183; Lebel et al; J. Allergy Clin. Immunol., 1988, 82, 869-877), bronchial asthma (Murray et al; N. Engl. J. Med., 1986, 315, 800-804; Liu et al; Am. Rev. Respir. Dis., 1990, 142, 126-132; Wenzel et al; J. Allergy Clin. Immunol., 1991, 87, 540-548), and urticaria (Heavey et al; J. Allergy Clin. Immunol., 1986, 78, 458-461). PGD₂ mediates it effects through two receptors, the PGD₂ (or DP) receptor (Boie et al; J. Biol. Chem., 1995, 270, 18910-18916) and the chemoattractant receptor-homologous molecule expressed on Th2 (or CRTH2) (Nagata et al; J. Immunol., 1999, 162, 1278-1289; Powell; Prostaglandins Luekot. Essent. Fatty Acids, 2003, 69, 179-185). Therefore, it has been postulated that agents that antagonise the effects of PGD₂ at its receptors may have beneficial effects in a number of disease states.

The CRTH2 receptor has been shown to be expressed on cell types associated with allergic inflammation, such as basophils, eosinophils, and Th2-type immune helper cells (Hirai et al; J. Exp. Med., 2001, 193, 255-261). The CRTH2 receptor has been shown to mediate PGD₂-mediated cell migration in these cell types (Hirai et al; J. Exp. Med., 2001, 193, 255-261), and also to play a major role in neutrophil and eosinophil cell recruitment in a model of contact dermatitis (Takeshita et al; Int. Immunol., 2004, 16, 947-959). Ramatroban {(3R)-3-[(4-fluorophenyl)sulphonylamino]-1,2,3,4-tetrahydro-9H-carbazole-9-propanoic acid}, a dual CRTH2 and thromboxane A₂ receptor antagonist, has been shown to attenuate these responses (Sugimoto et al; J. Pharmacol. Exp. Ther., 2003, 305, 347-352; Takeshita et al; op. cit.). The potential of PGD₂ both to enhance allergic inflammation and induce an inflammatory response has been demonstrated in mice and rats. Transgenic mice over expressing PGD₂ synthase exhibit an enhanced pulmonary eosinophilia and increased levels of Th2 cytokines in response to allergen challenge (Fujitani et al, J. Immunol., 2002, 168, 443-449). In addition, exogenously administered CRTH2 agonists enhance the allergic response in sensitised mice (Spik et al; J. Immunol., 2005, 174, 3703-3708). In rats exogenously applied CRTH2 agonists cause a pulmonary eosinophilia but a DP agonist (BW 245C) or a TP agonist (I-BOP) showed no effect (Shirashi et al; J. Pharmacol. Exp Ther., 2005, 312, 954-960). These observations suggest that CRTH2 antagonists may have valuable properties for the treatment of diseases mediated by PGD₂.

Our copending application PCT/GB2006/002341 relates to CRTH2 antagonist compounds of formula (I) and salts, N-oxides, hydrates and solvates thereof:

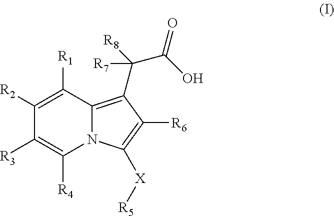

wherein

R₁, R₂, R₃ and R₄ each independently are hydrogen, C₁-C₆alkyl, fully or partially fluorinated C₁-C₆alkyl, halo, —S(O)ₙR₁₀, —SO₂N(R₁₀)₂, —N(R₁₀)₂, —C(O)N(R₁₀)₂, —NR₁₀C(O)R₉, —CO₂R₁₀, —C(O)R₉, —NO₂, —CN or —OR₁₁;
wherein each R₉ is independently C₁-C₆alkyl, aryl, heteroaryl;
R₁₀ is independently hydrogen, C₁-C₆alkyl, aryl, or heteroaryl;
R₁₁ is hydrogen, C₁-C₆alkyl, fully or partially fluorinated C₁-C₆alkyl or a group —SO₂R₉;
n is 0, 1 or 2;

R₅ is C₁-C₆alkyl, fully or partially fluorinated C₁-C6alkyl, C₁-C6alkenyl, C₁-C₆alkynyl, optionally substituted aryl, or optionally substituted heteroaryl;

R₆ is hydrogen, C₁-C₆alkyl or fully or partially fluorinated C₁-C₆alkyl;

R₇ and R₈ are independently hydrogen or C₁-C₆alkyl, or R₇ and R₈ together with the atom to which they are attached form a cycloalkyl group; and

X is —CHR₆—, —S(O)ₙ—, —NR₆SO₂— or —SO₂NR₆— wherein n is 0, 1 or 2,

PROVIDED THAT when X is —CH₂—, R₆ is methyl and R₅ is 4-chlorophenyl, then R₁, R₂, R₃, R₄, R₇ and R₈ are not all hydrogen.

### DETAILED DESCRIPTION OF THE INVENTION

The present invention provides a group of specific compounds falling within the scope of, but not specifically disclosed in our copending application PCT/GB2006/002341 referred to above.

The invention provides a compound selected from the group consisting of

[3-(2,4-dichlorophenylsulfanyl)-6-fluoro-2-methylindolizin-1-yl]acetic acid,

[6-fluoro-3-(2-fluoro-4-methanesulfonylphenylsulfanyl)-2-methylindolizin-1-yl]acetic acid,

[6-fluoro-3-(4-methanesulfonyl-2-trifluoromethylphenylsulfanyl)-2-methylindolizin-1-yl]acetic acid,

(R) 2-[6-fluoro-3-(4-methanesulfonylphenylsulfanyl)-2-methylindolizin-1-yl]propionic acid,

(S) 2-[6-fluoro-3-(4-methanesulfonylphenylsulfanyl)-2-methylindolizin-1-yl]propionic acid,

[3-(4-ethanesulfonylphenylsulfanyl)-6-fluoro-2-methylindolizin-1-yl]acetic acid,

ethanesulfonylaminobenzenesulfonyl)-6-fluoro-2-methylindolizin-1-yl]acetic acid,

[7-chloro-6-fluoro-3-(4-methanesufonylphenylsulfanyl)-2-methylindolizin-1-yl]acetic acid,

[3-(2-chloro-4-methanesulfonylphenylsulfanyl)-7-cyano-2-methylindolizin-1-yl]acetic acid,

[6-cyano-3-(4-methanesulfonylbenzyl)-2-methylindolizin-1-yl]acetic acid,

[3-(4-chlorobenzyl)-7-cyano-2-methylindolizin-1-yl]acetic acid,

[6-cyano-3-(6-fluoroquinolin-2-ylmethyl)-2-methylindolizin-1-yl]acetic acid,

[6-fluoro-3-(4-methoxyphenylsulfanyl)-2-methylindolizin-1-yl]acetic acid,

[7-chloro-6-fluoro-3-(4-methoxyphenylsulfanyl)-2-methylindolizin-1-yl]acetic acid,

[3-(4-bromophenylsulfanyl)-6-fluoro-2-methylindolizin-1-yl]acetic acid,

[3-(4-cyclopropylsulfamoylphenylsufanyl)-6-fluoro-2-methylindolizin-1-yl]acetic acid, and salts, N-oxides, hydrates and solvates thereof.

Compounds with which the invention is concerned are CRTH2 receptor antagonists.

A second aspect of the invention is a pharmaceutical composition comprising a compound of the invention in admixture with a pharmaceutically acceptable carrier or excipient.

A third aspect of the invention is a compound of the invention for use in therapy.

A fourth aspect of the invention is the use of a compound of the invention in the manufacture of a medicament for the treatment of a disease in which a CRTH2 antagonist can prevent, inhibit or ameliorate the pathology and/or symptomatology of the disease. Such diseases include asthma, chronic obstructive pulmonary disease, rhinitis, allergic airway syndrome, or allergic rhinobronchitis, as well as atopic and non-atopic dermatitis, Crohn's disease, ulcerative colitis, and irritable bowel disease.

A fifth aspect of the invention is a method for treating a disease in a patient in which a CRTH2 antagonist can prevent, inhibit or ameliorate the pathology and/or symptomatology of the disease, which method comprises administering to the patient a therapeutically effective amount of a compound of the invention.

In particular, compounds with which the invention is concerned are useful in the treatment of disease associated with elevated levels of prostaglandin D2 (PGD2) or one or more active metabolites thereof.

Examples of such diseases include asthma, rhinitis, allergic airway syndrome, allergic rhinobronchitis, bronchitis, chronic obstructive pulmonary disease (COPD), nasal polyposis, sarcoidosis, farmer's lung, fibroid lung, cystic fibrosis, chronic cough, conjunctivitis, atopic and non-atopic dermatitis, Alzheimer's disease, amyotrophic lateral sclerosis, AIDS dementia complex, Huntington's disease, frontotemporal dementia, Lewy body dementia, vascular dementia, Guillain-Barre syndrome, chronic demyelinating polyradiculoneurophathy, multifocal motor neuropathy, plexopathy, multiple sclerosis, encephalomyelitis, panencephalitis, cerebellar degeneration and encephalomyelitis, CNS trauma, migraine, stroke, rheumatoid arthritis, ankylosing spondylitis, Behcet's Disease, bursitis, carpal tunnel syndrome, inflammatory bowel disease, Crohn's disease, ulcerative colitis, dermatomyositis, Ehlers-Danlos Syndrome (EDS), fibromyalgia, myofascial pain, osteoarthritis (OA), osteonecrosis, psoriatic arthritis, Reiter's syndrome (reactive arthritis), sarcoidosis, scleroderma, Sjogren's Syndrome, soft tissue disease, Still's Disease, tendinitis, polyarteritis Nodossa, Wegener's Granulomatosis, myositis (polymyositis dermatomyositis), gout, atherosclerosis, lupus erythematosus, systemic lupus erythematosus (SLE), type I diabetes, nephritic syndrome, glomerulonephritis, acute and chronic renal failure, eosinophilia fascitis, hyper IgE syndrome, sepsis, septic shock, ischemic reperfusion injury in the heart, allograft rejection after transplantations, and graft versus host disease.

However, the compounds with which the invention is concerned are primarily of value for the treatment of asthma, chronic obstructive pulmonary disease, rhinitis, allergic airway syndrome, or allergic rhinobronchitis, as well as atopic and non-atopic dermatitis, Crohn's disease, ulcerative colitis, and irritable bowel disease.

As used herein the term “salt” includes base addition, acid addition and quaternary salts. Compounds of the invention which are acidic can form salts, including pharmaceutically acceptable salts, with bases such as alkali metal hydroxides, e.g. sodium and potassium hydroxides; alkaline earth metal hydroxides e.g. calcium, barium and magnesium hydroxides; with organic bases e.g. N-methyl-D-glucamine, choline tris(hydroxymethyl)amino-methane, L-arginine, L-lysine, N-ethyl piperidine, dibenzylamine and the like. Specific salts with bases include the benzathine, calcium, diolamine, meglumine, olamine, potassium, procaine, sodium, tromethamine and zinc salts. Those compounds of the invention which are basic can form salts, including pharmaceutically acceptable salts with inorganic acids, e.g. with hydrohalic acids such as hydrochloric or hydrobromic acids, sulphuric acid, nitric acid or phosphoric acid and the like, and with organic acids e.g. with acetic, tartaric, succinic, fumaric, maleic, malic, salicylic, citric, methanesulphonic, p-toluenesulphonic, benzoic, benzenesunfonic, glutamic, lactic, and mandelic acids and the like.

Use of prodrugs, such as esters, of compounds with which the invention is concerned is also part of the invention. “Prodrug” means a compound which is convertible in vivo by metabolic means (e.g. by hydrolysis, reduction or oxidation) to a compound of formula (I). For example an ester prodrug of a compound of formula (I) may be convertible by hydrolysis in vivo to the parent molecule. Suitable esters of compounds of formula (I) are for example acetates, citrates, lactates, tartrates, malonates, oxalates, salicylates, propionates, succinates, fumarates, maleates, methylene-bis-β-hydroxynaphthoates, gentisates, isethionates, di-p-toluoyltartrates, methanesulphonates, ethanesulphonates, benzenesulphonates, p-toluene-sulphonates, cyclohexylsulphamates and quinates. Examples of ester prodrugs are those described by F. J. Leinweber, Drug Metab. Res., 1987, 18, 379. As used in herein, references to the compounds of formula (I) are meant to also include the prodrug forms.

Compositions

As mentioned above, the compounds with which the invention is concerned are CRTH2 receptor antagonists, and are useful in the treatment of diseases which benefit from such modulation. Examples of such diseases are referred to above, and include asthma, rhinitis, allergic airway syndrome, and allergic rhinobronchitis.

It will be understood that the specific dose level for any particular patient will depend upon a variety of factors including the activity of the specific compound employed, the age, body weight, general health, sex, diet, time of administration, route of administration, rate of excretion, drug combination and the severity of the particular disease undergoing treatment. Optimum dose levels and frequency of dosing will be determined by clinical trial, as is required in the pharmaceutical art. In general, the daily dose range will lie within the range of from about 0.001 mg to about 100 mg per kg body weight of a mammal, often 0.01 mg to about 50 mg per kg, for example 0.1 to 10 mg per kg, in single or divided doses. On the other hand, it may be necessary to use dosages outside these limits in some cases.

The compounds with which the invention is concerned may be prepared for administration by any route consistent with their pharmacokinetic properties. Orally administrable compositions may be in the form of tablets, capsules, powders, granules, lozenges, liquid or gel preparations, such as oral, topical, or sterile parenteral solutions or suspensions. Tablets and capsules for oral administration may be in unit dose presentation form, and may contain conventional excipients such as binding agents, for example syrup, acacia, gelatin, sorbitol, tragacanth, or polyvinyl-pyrrolidone; fillers for example lactose, sugar, maize-starch, calcium phosphate, sorbitol or glycine; tabletting lubricant, for example magnesium stearate, talc, polyethylene glycol or silica; disintegrants for example potato starch, or acceptable wetting agents such as sodium lauryl sulphate. The tablets may be coated according to methods well known in normal pharmaceutical practice. Oral liquid preparations may be in the form of, for example, aqueous or oily suspensions, solutions, emulsions, syrups or elixirs, or may be presented as a dry product for reconstitution with water or other suitable vehicle before use. Such liquid preparations may contain conventional additives such as suspending agents, for example sorbitol, syrup, methyl cellulose, glucose syrup, gelatin hydrogenated edible fats; emulsifying agents, for example lecithin, sorbitan monooleate, or acacia; non-aqueous vehicles (which may include edible oils), for example almond oil, fractionated coconut oil, oily esters such as glycerine, propylene glycol, or ethyl alcohol; preservatives, for example methyl or propyl p-hydroxybenzoate or sorbic acid, and if desired conventional flavouring or colouring agents.

For topical application to the skin, the drug may be made up into a cream, lotion or ointment. Cream or ointment formulations which may be used for the drug are conventional formulations well known in the art, for example as described in standard textbooks of pharmaceutics such as the British Pharmacopoeia.

The drug may also be formulated for inhalation, for example as a nasal spray, or dry powder or aerosol inhalers. For delivery by inhalation, the active compound is preferably in the form of microparticles. They may be prepared by a variety of techniques, including spray-drying, freeze-drying and micronisation. Aerosol generation can be carried out using, for example, pressure-driven jet atomizers or ultrasonic atomizers, preferably using propellant-driven metered aerosols or propellant-free administration of micronized active compounds from, for example, inhalation capsules or other “dry powder” delivery systems.

The active ingredient may also be administered parenterally in a sterile medium. Depending on the vehicle and concentration used, the drug can either be suspended or dissolved in the vehicle. Advantageously, adjuvants such as a local anaesthetic, preservative and buffering agents can be dissolved in the vehicle.

Other compounds may be combined with compounds of this invention for the prevention and treatment of prostaglandin-mediated diseases. Thus the present invention is also concerned with pharmaceutical compositions for preventing and treating PGD₂-mediated diseases comprising a therapeutically effective amount of a compound of the invention and one or more other therapeutic agents. Suitable therapeutic agents for a combination therapy with compounds of the invention include, but are not limited to: (1) corticosteroids, such as fluticasone, budesonide or ciclesonide; (2) β2-adrenoreceptor agonists, such as salmeterol, formeterol or indacaterol; (3) leukotriene modulators, for example leukotriene antagonists such as montelukast or pranlukast or leukotriene biosynthesis inhibitors such as Zileuton or BAY-x1005; (4) anticholinergic agents, for example muscarinic-3 (M₃) receptor antagonists such as tiotropium bromide; (5) phosphodiesterase-IV (PDE-IV) inhibitors, such as roflumilast or cilomilast; (6) antihistamines, for example selective histamine-1 (H1) receptor antagonists, such as loratidine or astemizole; (7) antitussive agents, such as codeine or dextramorphan; (8) non-selective COX-1/COX-2 inhibitors, such as ibuprofen or ketoprofen; (9) COX-2 inhibitors, such as celecoxib and rofecoxib; (10) VLA-4 antagonists, such as those described in WO97/03094 and WO97/02289; (11) TNF-α inhibitors, for example anti-TNF monoclonal antibodies, such as Remicade and CDP-870 and TNF receptor immunoglobulin molecules, such as Enbrel; (12) inhibitors of matrix metalloprotease (MMP), for example MMP8, 9 and 12; (13) human neutrophil elastase inhibitors, such as those described in WO2005/026124 and WO2003/053930; (14) Adenosine A2a agonists such as those described in EP1052264 and EP1241176 (15) Adenosine A2b antagonists such as those described in WO2002/42298; (16) modulators of chemokine receptor function, for example antagonists of CCR3 and CCR8; (17) compounds which modulate the action of other prostanoid receptors, for example a PGD2 (DP) receptor antagonist or a thromboxane A2 antagonist; and (18) compounds which modulate Th2 function, for example, PPAR agonists.

The weight ratio of the compound of the invention to the second active ingredient may be varied and will depend upon the effective dose of each ingredient. Generally, an effective dose of each will be used.

Biological Methods

Compounds of the invention can be tested using the following biological test methods to determine their ability to displace PGD₂ from the CRTH2 receptor and for their ability to antagonise the functional effects of PGD₂at the CRTH2 receptor in a whole cell system.

Radioligand Binding Assay

The receptor binding assay is performed in a final volume of 200 μL binding buffer [10 mM BES (pH 7.4), 1 mM EDTA, 10 mM manganese chloride, 0.01% BSA] and 1 nM [³H]-PGD₂ (Amersham Biosciences UK Ltd). Ligands are added in assay buffer containing a constant amount of DMSO (1% by volume). Total binding is determined using 1% by volume of DMSO in assay buffer and non-specific binding is determined using 10 μM of unlabeled PGD₂ (Sigma). Human embryonic kidney (HEK) cell membranes (3.5 μg) expressing the CRTH2 receptor are incubated with 1.5 mg wheatgerm agglutinin SPA beads and 1 nM [³H]-PGD₂ (Amersham Biosciences UK Ltd) and the mixture incubated for 3 hours at room temperature. Bound [³H]-PGD₂ is detected using a Microbeta TRILUX liquid scintillation counter (Perkin Elmer). Compound IC₅₀ value is determined using a 6-point dose response curve in duplicate with a semi-log compound dilution series. IC₅₀ calculations are performed using Excel and XLfit (Microsoft), and this value is used to determine a Ki value for the test compound using the Cheng-Prusoff equation.

Functional Assay

Calcium Mobilisation (Flux) Assay

Stable CHO-K1 cells co-expressing the CRTH2 receptor and the G-protein Gα16 are seeded (40,000 cells per well in a plating volume of 75 μL in F-12 Hams supplemented with 1% foetal bovine serum) into collagen-coated 96-well plates 24 hours prior to the assay. The cells are then loaded with a fluorescence-imaging plate reader (FLIPR) calcium kit dye (Calcium 3 kit, Molecular Devices Ltd) containing 5 mM final concentration of probenecid and incubated at 37° C. for 1 hour in a 5 % CO₂ atmosphere. The fluorescence emission caused by intracellular calcium mobilization elicited by the PGD₂ at the CRTH2 receptor is determined with a FLEXstation benchtop scanning and integrated fluid transfer workstation (Molecular Devices Ltd). To detect antagonists and determine compound IC₅₀, compounds are pre-incubated at varying concentrations with the loaded cells for 15 minutes at 37° C., 5 % CO₂, prior to the addition of the agonist at its EC₈₀ value. Compounds and agonist are added in Hanks balanced salt solution containing 20 mM HEPES and 0.1% BSA). The fractional response values for each well are calculated by subtracting the basal response from the peak response. Results are calculated as the mean of triplicate wells using Excel and XLfit (Microsoft).

The following examples describe the preparation of compounds of the invention:

### Examples

¹H NMR spectra were recorded at ambient temperature using a Varian Unity Inova (400 MHz) spectrometer with a triple resonance 5 mm probe spectrometer. Chemical shifts are expressed in ppm relative to tetramethylsilane. The following abbreviations have been used: br s=broad singlet, s=singlet, d=doublet, dd=double doublet, t=triplet, q=quartet, m=multiplet.

Mass Spectrometry (LCMS) experiments to determine retention times and associated mass ions were performed using the following methods:

Method A: experiments were performed on a Micromass Platform LCT spectrometer with positive ion electrospray and single wavelength UV 254 nm detection using a Higgins Clipeus C18 5 μm 100×3.0 mm column and a 2 mL/minute flow rate. The initial solvent system was 95% water containing 0.1% formic acid (solvent A) and 5% acetonitrile containing 0.1% formic acid (solvent B) for the first minute followed by a gradient up to 5% solvent A and 95% solvent B over the next 14 minutes. The final solvent system was held constant for a further 2 minutes.

Method B: experiments were performed on a Micromass Platform LC spectrometer with positive and negative ion electrospray and ELS/Diode array detection using a Phenomenex Luna C18(2) 30×4.6 mm column and a 2 mL/minute flow rate. The solvent system was 95% solvent A and 5% solvent B for the first 0.50 minutes followed by a gradient up to 5% solvent A and 95% solvent B over the next 4 minutes. The final solvent system was held constant for a further 0.50 minutes Microwave experiments were carried out using a Personal Chemistry Smith Synthesizer™, which uses a single-mode resonator and dynamic field tuning, both of which give reproducibility and control. Temperatures from 40-250° C. can be achieved, and pressures of up to 20 bar can be reached. Two types of vial are available for this processor, 0.5-2.0 mL and 2.0-5.0 mL.

Reverse-phase preparative HPLC purifications were carried out using Genesis 7 micron C-18 bonded silica stationary phase in columns 10 cm in length and 2 cm Internal diameter. The mobile phase used was mixtures of acetonitrile and water (both buffered with 0.1% v/v trifluoroacetic acid) with a flow rate of 10 mL per minute and typical gradients of 40 to 90% organic modifier ramped up over 30 to 40 minutes. Fractions containing the required product (identified by LC-MS analysis) were pooled, the organic fraction removed by evaporation, and the remaining aqueous fraction lyophilised, to give the final product.

### Example 1

### [3-(2,4dichlorophenylsulfanyl)-6-fluoro-2-methylindolizin-1-yl]acetic acid

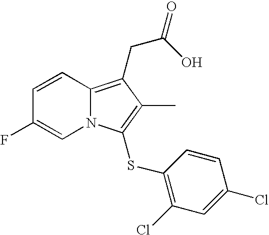

### Preparation 1a: 3-(5-fluoropyridin-2-yl)propionic acid ethyl ester

A solution of 3-ethoxy-3-oxopropylzinc bromide in tetrahydrofuran (0.5 M, 630 mL) was added dropwise to a mixture of 2-bromo-5-fluoropyridine (50 g), tetrakis(triphenylphosphine)palladium(0), (5.5 g) and toluene (350 mL) at room temperature. The resulting mixture was stirred at room temperature for 24 hours and then concentrated under reduced pressure. The residue was diluted with ethyl acetate, filtered through hyflo and the filtrate washed with saturated aqueous sodium chloride solution and dried over magnesium sulfate. The solvent was removed under reduced pressure and the residue purified by column chromatography on silica gel, eluting with a mixture of pentane and ethyl acetate (9:1 by volume) to afford title compound as a yellow oil, 35 g.

¹H NMR (CDCl₃): δ 1.25 (t, J=7.1 Hz, 3H), 2.75 (t, J=7.4 Hz, 2H), 3.10 (t, J=7.4 Hz, 2H), 4.10 (q, J=7.1 Hz, 2H), 7.20 (dd, J=4.4, 8.4 Hz, 1H), 7.30 (dt, J=3.0, 8.4 Hz, 1H), 8.35 (d, J=2.6 Hz, 1H).

### Preparation 1b: (6-fluoro-2-methylindolizin-1-yl)acetic acid ethyl ester

A mixture of 3-(5-fluoropyridin-2-yl)propionic acid ethyl ester (12 g) and acetonitrile (90 mL) at room temperature was treated with a solution of 1-bromopropan-2-one (16 g) in acetonitrile (30 mL) and the resulting mixture heated at reflux for 24 hours and then left to stand at room temperature for 60 hours. The mixture was treated with pyridine (35 mL) and the resulting mixture was heated at reflux for four hours, cooled to room temperature and then concentrated under reduced pressure. The residue was diluted with ethyl acetate, washed with water and saturated aqueous sodium hydrogen chloride solution and dried over magnesium sulfate. The solvent was removed under reduced pressure and the residue purified by column chromatography on silica gel, eluting with a mixture of pentane and ethyl acetate (19:1 by volume) to afford title compound as a yellow oil, 3.7 g.

¹H NMR (CDCl₃): δ 1.25 (t, J=7.1 Hz, 3H), 2.25 (s, 3H), 3.65 (s, 2H), 4.11 (q, J=7.1 Hz, 2H), 6.55 (m, 1H), 7.10 (br s, 1H), 7.25 (m, 1H), 7.70 (d, J=3.4 Hz, 1H).

### Preparation 1c: [3-(2,4-dichlorophenylsulfanyl)-6-fluoro-2-methylindolizin-1-yl]acetic acid ethyl ester

A mixture of (6-fluoro-2-methylindolizin-1-yl)acetic acid ethyl ester (0.10 g) and toluene (20 mL) was treated with 2,4-dichlorobenzenesulfonyl chloride (0.16 g) and aluminium chloride (0.010 g) and the resulting mixture was heated at reflux for 24 hours. The mixture was cooled to room temperature, concentrated under reduced pressure and the residue diluted with a mixture of ethyl acetate and water. The mixture was filtered through hyflo and the organic filtrate dried over magnesium sulfate. The solvent was removed under reduced pressure and the residue purified by column chromatography on silica gel, eluting with a mixture of pentane and ethyl acetate (19:1 by volume) to afford title compound as a light brown oil, 0.052 g.

¹H NMR (CDCl₃): δ 1.25 (t, J=7.1 Hz, 3H), 2.35 (s, 3H), 3.75 (s, 2H), 4.15 (q, J=7.1 Hz, 2H) 6.00 (d, J=8.5 Hz,₁ H), 6.80 (m, 1H), 6.90 (d, J=2.1 Hz, 1H), 7.35 (d, J=2.1 Hz, 1H), 7.40 (dd, J=5.2, 9.7 Hz, 1H), 8.00 (dd, J=2.1, 5.2 Hz, 1H)

### Preparation 1d: [3-(2,4-dichlorophenylsulfanyl)-6-fluoro-2-methylindolizin-1-yl]acetic acid

A mixture of [3-(2,4-dichlorophenylsulfanyl)-6-fluoro-2-methylindolizin-1-yl]acetic acid ethyl ester (0.052 g) and methanol (2.0 mL) was treated with 1.0 M aqueous lithium hydroxide solution and the resulting mixture was stirred at room temperature for 2 hours. The mixture was concentrated under reduced pressure, diluted with water and acidified by the addition of 1.0 M aqueous hydrochloric acid. The mixture was extracted with dichloromethane and the combined extracts dried over magnesium sulfate and the solvent removed under reduced pressure. The residue was purified by preparative reverse-phase HPLC using a gradient over 30 minutes of acetonitrile in water (70% to 95% of organic modifier) to afford title compound as a white solid, 0.032 g.

¹H NMR (CDCl₃): δ 2.35 (s, 3H), 3.80 (s, 2H), 6.00 (d, J=8.5 Hz, 1H), 6.80 (m, 1H), 6.90 (dd, J=2.1, 8.5 Hz, 1H), 7.35 (d, J=1.9 Hz, 1H), 7.40 (m, 1H), 8.00 (dd, J=1.9, 5.0 Hz, 1H).

MS: ESI (+ve) (Method A): 384 (M+H)⁺, Retention time 14.0 min.

### Example 2

### [6-fluoro-3-(2-fluoro-4-methanesulfonylphenylsulfanyl)-2-methylindolizin-1-yl]acetic acid

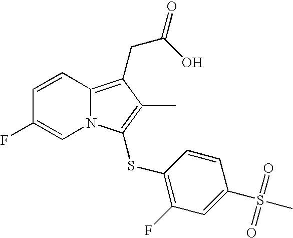

### Preparation 2a: 1,2-difluoro-4-methanesulfonylbenzene

3-Chlordperoxybenzoic acid (19 g) was added portionwise to a solution of 1,2-difluoro-4-methylsulfanylbenzene (5.4 g) in dichloromethane (100 mL) at room temperature and the resulting mixture was stirred at room temperature overnight. The mixture was washed with saturated aqueous sodium thiosulfate solution and saturated aqueous sodium hydrogen carbonate solution and then dried over magnesium sulfate. The solvent was removed under reduced pressure to afford title compound as a white solid, 5.5 g.

¹H NMR (CDCl₃): δ 3.05 (s, 3H), 7.40 (m, 1H), 7.75-7.85 (m, 2H).

### Preparation 2b: bis(2-fluoro-4-methanesulfonylbenzene)disulfide

A mixture of 1,2-difluoro-4-methanesulfonylbenzene (2.8 g), sodium hydrogensulfide (11 g) and 1-methylpyrrolidin-2-one (30 mL) was stirred at 80° C. for 90 minutes and then at room temperature overnight. The mixture was diluted with water (40 mL), acidified by the addition of concentrated hydrochloric acid and then extracted with ethyl acetate. The combined extracts were dried over magnesium sulfate and the solvent removed under reduced pressure. The residue was crystallised from toluene to afford title compound as cream crystals, 1.4 g.

MS: ESI (+ve) (Method B): 411 (M+H)⁺, Retention time 3.1 min.

### Preparation 2c: [6-fluoro-3-(2-fluoro-4-methanesulfonylphenylsulfanyl)-2-methylindolizin-1-yl]acetic acid ethyl ester

Sulfuryl chloride (0.066 mL) was added to a mixture of bis(2-fluoro-4-methanesulfonylbenzene)disulfide (0.39 g) and dichloroethane (5.0 mL) at 0° C. and the resulting mixture was stirred at room temperature for 30 minutes. A solution of (6-fluoro-2-methylindolizin-1-yl)acetic acid ethyl ester (0.15 g) in dichloroethane (5.0 mL) was added and the resulting mixture stirred at room temperature overnight. The mixture was concentrated under reduced pressure and the residue purified by column chromatography on silica gel, eluting with mixture of toluene, dichloromethane and ethyl acetate (1:4:0 to 0:1:0 to 0:9:1 by volume) to afford title compound as a yellow solid, 0.25 g.

MS: ESI (+ve) (Method B): 440 (M+H)⁺, Retention time 4.1 min.

### Preparation 2d: 6-fluoro-3-(2-fluoro-4-methanesulfonylphenylsulfanyl)-2-methylindolizin-1-yl]acetic acid

A mixture of [6-fluoro-3-(2-fluoro-4-methanesulfonylphenylsulfanyl)-2-methylindolizin-1-yl]acetic acid ethyl ester (0.25 g), tetrahydrofuran (3.0 mL) and 1.0 M aqueous lithium hydroxide solution (3.0 mL) was stirred at room temperature for 3 hours. The tetrahydrofuran was removed under reduced pressure and the pH of the residue adjusted to 4 by the addition of sodium dihydrogenphosphate. The resulting precipitate was collected by filtration to afford title compound, 0.22 g.

¹H NMR (DMSO-d6): 2.25 (s, 3H), 3.20 (s, 3H), 3.70 (s, 2H), 6.40 (dd, J=7.5, 8.1 Hz, 1H), 7.00 (m, 1H), 7.55 (dd, J=1.8, 8.3 Hz, 1H), 7.70 (dd, J=5.5, 9.8 Hz, 1H), 7.85 (dd, J=1.8, 7.5 Hz, 1H), 8.35 (dd, J=2.1, 5.5 Hz, 1H).

MS: ESI (+ve) (Method A): 412 (M+H)⁺, Retention time 10.2 min.

### Example 3

### [6-fluoro-3-(4-methanesulfonyl-2-trifluoromethylphenylsulfanyl)-2-methylindolizin-1-yl]acetic acid

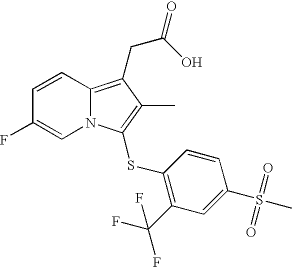

### Preparation 3a: 1-fluoro-4-methylsulfanyl-2-trifluoromethylbenzene

A mixture of dimethyl disulfide (2.0 mL), 4-fluoro-3-trifluoromethylphenylamine (2.0 g) and acetonitrile (50 mL) at room temperature was treated dropwise with isopentyl nitrite (2.0 mL) and the resulting mixture was stirred at 60° C. for 16 hours. The mixture was cooled to room temperature, diluted with water and extracted with ethyl acetate. The combined extracts were dried over magnesium sulfate and the solvent was removed under reduced pressure to afford title compound, 0.67 g.

¹H NMR (CDCl₃): δ 2.50 (s, 3H), 7.15 (m, 1H), 7.40-7.50 (m, 2H).

### Preparation 3b: 1-fluoro-4-methanesulfonyl-2-trifluoromethylbenzene

3-Chloroperoxybenzoic acid (2.4 g) was added portionwise to a solution of 1-fluoro-4-methylsulfanyl-2-trifluoromethylbenzene (1.3 g) in dichloromethane (50 mL) at room temperature and the resulting mixture was stirred at room temperature for 3 hours. The mixture was filtered and the filtrated washed with saturated aqueous sodium thiosulfate solution and saturated aqueous sodium chloride solution and then dried over magnesium sulfate. The solvent was removed under reduced pressure to afford title compound, 1.5 g.

¹H NMR (CDCl₃): δ 3.1 (s, 3H), 7.45 (t, J=9.3 Hz, 1H), 8.20 (m, 1H), 8.25 (m, 1H).

### Preparation 3c: bis(2-trifluoromethyl-4-methanesulfonylbenzene)disulfide

A mixture of 1-fluoro-4-methanesulfonyl-2-trifluoromethylbenzene (1.4 g), sodium hydrogensulfide (4.6 g) and 1-methylpyrrolidin-2-one (6.0 mL) was stirred at 80° C. for 2 hours and then at room temperature overnight. The mixture was diluted with water, washed with ethyl acetate and the aqueous phase acidified by the addition of concentrated hydrochloric acid. The mixture was extracted with ethyl acetate and the combined extracts dried over magnesium sulfate and the solvent removed under reduced pressure. The residue was triturated with ethyl acetate to afford title compound, 0.94 g.

### Preparation 3d: [6-fluoro-3-(2-trifluoromethyl-4-methanesulfonylphenylsulfanyl)-2-methylindolizin-1-yl]acetic acid ethyl ester

Sulfuryl chloride (0.060 mL) was added to a mixture of bis(2-trifluoromethyl-4-methanesulfonylbenzene)disulfide (0.45 g) and dichloroethane (10 mL) at 0° C. and the resulting mixture was stirred at room temperature for 30 minutes and then at 60° C. for 4 hours. A solution of (6-fluoro-2-methylindolizin-1-yl)acetic acid ethyl ester (0.15 g) in dichloroethane (5.0 mL) was added and the resulting mixture stirred 60° C. overnight. The mixture was concentrated under reduced pressure and the residue purified by column chromatography on silica gel, eluting with mixture of cyclohexane, dichloromethane and ethyl acetate (1:4:0 to 0:19:1 by volume). Further purification by preparative reverse-phase HPLC using a gradient over 20 minutes of acetonitrile. in water (50% to 98% of organic modifier) gave title compound as a yellow solid, 0.030 g.

MS: ESI (+ve) (Method B): 490 (M+H)⁺, Retention time 4.3 min.

### Preparation 3e: [6-fluoro-3-(4-methanesulfonyl-2-trifluoromethylphenylsulfanyl)-2-methylindolizin-1-yl]acetic acid

The title compound was prepared by the method of Preparation 2d using [6-fluoro-3-(2-fluoro-4-methanesulfonylphenylsulfanyl)-2-methylindolizin-1-yl]acetic acid ethyl ester

¹H NMR (DMSO-d6): δ 2.25 (s, 3H), 3.25 (s, 3H), 3.80 (s, 2H), 6.50 (d, J=8.5 Hz, 1H), 7.10 (m, 1H), 7.70 (dd, J=5.5, 9.8 Hz, 1H), 7.90 (dd, J=1.9, 8.4 Hz, 1H), 8.20 (d, J=1.9 Hz, 1H), 8.20 (m, 1H), 12.20 (br s, 1H).

MS: ESI (+ve) (Method A): 462 (M+H)⁺, Retention time 11.1 min.

### Example 4

### (R)-2-[6-fluoro-3-(4-methanesulfonylphenylsulfanyl)-2-methylindolizin-1-yl]propionic acid

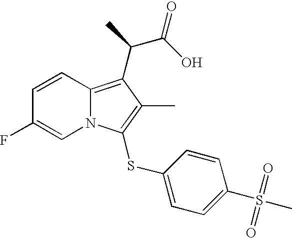

### Preparation 4a: (R)-3-(5-fluoropyridin-2-yl)-2-methylpropionic acid methyl ester

A solution of (S)-3-methoxy-2-methyl-3-oxopropylzinc bromide in tetrahydrofuran (0.5 M, 20 mL) was added dropwise to a mixture of 2-bromo-5-fluoropyridine (1.0 g), tetrakis(triphenylphosphine)palladium(0), (0.14 g) and toluene (20 mL) at room temperature. The resulting mixture was stirred at room temperature for 6 hours and then left to stand at room temperature overnight. The mixture was concentrated under reduced pressure and the residue diluted with a mixture of ethyl acetate and water. The mixture was filtered through hyflo and the organic filtrate washed with water and saturated aqueous sodium chloride solution and then dried over magnesium sulfate. The solvent was removed under reduced pressure and the residue purified by column chromatography on silica gel, eluting with a mixture of pentane and ethyl acetate (9:1 by volume) to afford title compound, 0.87 g.

¹H NMR (CDCl₃): δ 1.20 (d, J=7.0 Hz, 3H), 2.85 (dd, J=6.6, 13.7 Hz, 1H), 3.00 (m, 1H), 3.20 (dd, J=7.6, 13.7 Hz, 1H), 3.65 (s, 3H), 7.15 (dd, J=4.4, 8.5 Hz, 1H), 7.30 (dt, J=2.9, 8.5 Hz, 1H), 8.40 (d, J=2.9 Hz, 1H).

### Preparation 4b: (R)-2-(6-fluoro-2-methylindolizin-1-yl)propionic acid methyl ester

The title compound was prepared by the method of Preparation 1b using (R)-3-(5-fluoropyridin-2-yl)-2-methylpropionic acid methyl ester.

MS: ESI (+ve) (Method B): 236 (M+H)⁺, Retention time 3.7 min.

### Preparation 4c: bis[4-(methylsulfonyl)phenyl]disulfide

A mixture of 1-fluoro-4-methanesulfonylbenzene (32 g), sodium hydrogensulfide (64 g) and 1-methylpyrrolidin-2-one (100 mL) was stirred at 80° C. for 60 minutes and then at room temperature for 60 minutes. The mixture was diluted with water, filtered and the filtrate acidified by the addition of concentrated hydrochloric acid. The resulting precipitate was collected by filtration, washed with water and dried to afford title compound as a white solid, 15 g.

MS: ESI (+ve) (Method B): 375 (M+H)⁺, Retention time 3.3 min.

### Preparation 4d: (R)-2-[6-fluoro-3-(4-methanesulfonylphenylsulfanyl)-2-methylindolizin-1-yl]propionic acid methyl ester

The title compound was prepared by the method of Preparation 2c using (R)-3-(5-fluoropyridin-2-yl)-2-methylpropionic acid methyl ester and bis[4-(methylsulfonyl)phenyl]disulfide.

MS: ESI (+ve) (Method B): 422 (M+H)⁺, Retention time 4.0 min.

### Preparation 4e: (R)-2-[6-fluoro-3-(4-methanesulfonylphenylsulfanyl)-2-methylindolizin-1-yl]propionic acid

The title compound was prepared by the method of Preparation 1d using (R)-2-[6-fluoro-3-(4-methanesulfonylphenylsulfanyl)-2-methylindolizin-1-yl]propionic acid methyl ester

¹H NMR (CDCl₃): δ 1.60 (d, J=7.1 Hz, 3H), 2.35 (s, 3H), 3.00 (s, 3H), 4.10 (q, J=7.1 Hz, 1H), 6.80 (m, 1H), 6.90 (d, J=8.6 Hz, 2H), 7.50 (dd, J=5.3, 9.7 Hz, 1H), 7.70 (d, J=8.6 Hz, 2H), 8.00 (m, 1H).

MS: ESI (+ve) (Method A): 408 (M+H)⁺, Retention time 10.4 min.

### Example 5

### [3-(4-ethanesulfonylphenylsulfanyl)+fluoro-2-methylindolizin-1-yl]acetic acid

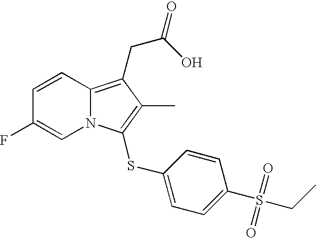

### Preparation 5a: 1-ethylsulfanyl-4-fluorobenzene

A mixture of diethyl disulfide (11 mL), 4-fluorophenylamine (5.0 g) and dichloromethane (50 mL) at room temperature was treated dropwise with tert-butyl nitrite (7.7 mL) and the resulting mixture stirred at 50° C. for 5 minutes and then at room temperature for 3 hours. The mixture was washed with water and 1.0 M aqueous hydrochloric acid and then dried over magnesium sulfate. The solvent was removed under reduced pressure and the residue purified by column chromatography on silica gel, eluting with cyclohexane to afford title compound, 3.5

¹H NMR (CDCl₃): δ 1.25 (t, J=7.3 Hz, 3H), 2.90 (q, J=7.3 Hz, 2H), 7.00 (m, 2H), 7.35 (m, 2H).

### Preparation 5b: 1-ethanesulfonyl-4-fluorobenzene

A solution of 1-ethylsulfanyl-4-fluorobenzene (3.5 g) in dichloromethane (20 mL) at room temperature was treated with 3-chloroperoxybenzoic acid (6.0 g) and the resulting mixture was stirred at room temperature overnight. The mixture was washed with saturated aqueous sodium hydrogen carbonate solution, dried over magnesium sulfate and the solvent removed under reduced pressure to afford title compound, 4.3 g.

¹H NMR (DMSO-d6): δ 1.10 (t, J=7.3 Hz, 3H), 3.30 (q, J=7.3 Hz, 2H), 7.50 (m, 2H), 8.00 (m, 2H).

### Preparation 5c: bis(4-ethanesultonylbenzene)disulfide

A mixture of 1-ethanesulfonyl-4-fluorobenzene (1.0 g), sodium hydrogensulfide (4.4 g) and 1-methylpyrrolidin-2-one (4.0 mL) was stirred at 80° C. for 2 hours and then at room temperature overnight. The mixture was diluted with water, washed with ethyl acetate and the aqueous phase acidified by the addition of 1.0 M aqueous hydrochloric acid. The mixture was extracted with ethyl acetate and the combined extracts dried over magnesium sulfate and the solvent removed under reduced pressure to afford title compound, 0.80 g.

¹H NMR (DMSO-d6): δ 1.10 (t, J=7.4 Hz, 6H), 3.30 (q, J=7.4 Hz, 4H), 7.80-7.90 (m, 8H).

### Preparation 5d: [3-(4-ethanesulfonylphenylsulfanyl)-6-fluoro-2-methylindolizin-1-yl]acetic acid ethyl ester

The title compound was prepared by the method of Preparation 2c using (6-fluoro-2-methylindolizin-1-yl)acetic acid ethyl ester and bis(4-ethanesulfonylbenzene)disulfide.

MS: ESI (+ve) (Method B): 436 (M+H)⁺, Retention time 4.1 min.

### Preparation 5e: [3-(4-ethanesulfonylphenylsulfanyl)-6-fluoro-2-methylindolizin-1-yl]acetic acid

A solution of [3-(4-ethanesulfonylphenylsulfanyl)-6-fluoro-2-methylindolizin-1-yl]acetic acid ethyl ester (0.14 g) in tetrahydrofuran (6.0 mL) was treated with a solution of lithium hydroxide (0.040 g) in water (6.0 mL) and the resulting mixture was stirred at room temperature for 1 hour. The mixture was acidified by the addition of 1.0 M aqueous hydrochloric acid, extracted with ethyl acetate and the combined extracts concentrated under reduce pressure. The residue was purified by preparative reverse-phase HPLC using a gradient of acetonitrile in water to afford title compound as a green solid, 0.020 g.

¹H NMR (CD₃OD): δ 1.15 (t, J=7.5 Hz, 3H), 2.30(s, 3H), 3.10 (q, J=7.5 Hz, 2H), 3.75 (s, 2H), 6.85 (m, 1H), 7.00 (d, J=8.7 Hz, 2H), 7.55 (m, 1H), 7.70 (d, J=8,7 Hz, 2H), 8.10 (dd, J=2.1, 5.4 Hz, 1H).

MS: ESI (+ve) (Method A): 435 (M+H)⁺, Retention time 10.2 min.

### Example 6

### (S)-2-[6-fluoro-3-(4-methanesulfonylphenylsulfanyl)-2-methylindolizin-1-yl]propionic acid

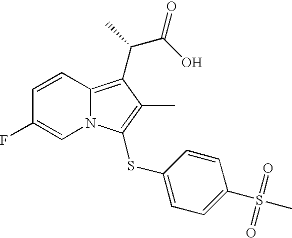

### Preparation 6a: (S)-3-(5-fluoropyridin-2-yl)-2-methylpropionic acid methyl ester

The title compound was prepared by the method of Preparation 4a using (R)-3-methoxy-2-methyl-3-oxopropylzinc bromide and 2-bromo-5-fluoropyridine.

MS: ESI (+ve) (Method B): 198 (M+H)⁺, Retention time 2.6 min.

### Preparation 6b: (S)-2-(6-fluoro-2-methylindolizin-1-yl)propionic acid methyl ester

The title compound was prepared by the method of Preparation 1b using (S)-3-(5-fluoropyridin-2-yl)-2-methylpropionic acid methyl ester.

MS: ESI (+ve) (Method B): 236 (M+H)⁺, Retention time 3.7 min.

### Preparation 6c: (S)-2-[6-fluoro-3-(4-methanesulfonylphenylsulfanyl)-2-methylindolizin-1-yl]propionic acid methyl ester

The title compound was prepared by the method of Preparation 2c using (S)-2-(6-fluoro-2-methylindolizin-1-yl)propionic acid methyl ester and bis[4-(methylsulfonyl)phenyl]disulfide.

MS: ESI (+ve) (Method B): 422 (M+H)⁺, Retention time 4.0 min.

### Preparation 6d: (S)-2-[6-fluoro-3-(4-methanesulfonylphenylsulfanyl)-2-methylindolizin-1-yl]propionic acid methyl ester

A mixture of (S)-2-[6-fluoro-3-(4-methanesulfonylphenylsulfanyl)-2-methylindolizin-1-yl]propionic acid methyl ester (0.25 g), methanol (10 mL) and 1.0 M aqueous lithium hydroxide solution (3.0 mL) was stirred at 65° C. for 8 hours. The mixture was concentrated under reduced pressure, diluted with water and washed with dichloromethane. The aqueous phase was acidified by the addition of glacial acetic acid, extracted with dichloromethane and the combined extracts dried over magnesium sulfate. The solvent was removed under reduced pressure and the residue purified by preparative reverse-phase HPLC using a gradient over 30 minutes of acetonitrile in water (50% to 98% of organic modifier) to afford title compound as a pale green solid, 0.17 g.

¹H NMR (CDCl₃): δ 1.60 (d, J=7.4 Hz, 3H), 2.35 (s, 3H), 3.00 (s, 3H), 4.05 (q, J=7.4 Hz, 1H), 6.75 (m, 1H), 6.90 (d, J=8.6 Hz, 2H), 7.50 (dd, J=5.3, 9.7 Hz, 1H), 7.70 (d, J=8.6 Hz, 2H), 8.80 (dd, J=2.0, 4.5 Hz, 1H).

MS: ESI (+ve) (Method A): 408 (M+H)⁺, Retention time 10.6 min.

### Example 7

### ethanesulfonylaminobenzenesulfonyl)-6-fluoro-2-methylindolizin-1-yl]acetic acid

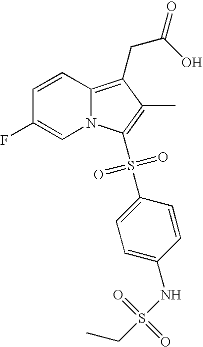

### Preparation 7a: ethanesulfonic acid [4-(4-ethanesulfonylaminophenyldisulfanyl)phenyl]amide

A solution of ethanesulfonyl chloride (1.9 mL) in dichloromethane (5.0 mL) was added dropwise to a solution of 4-aminophenyl disulfide (2.0 g) and triethylamine (4.5 mL) in dichloromethane (20 mL) at −40° C. and the resulting mixture was warmed to room temperature over a period of 4 hours. The mixture was washed with 1.0 M aqueous sodium hydroxide solution and the aqueous phase acidified by the addition of 1.0 M aqueous hydrochloric acid and then extracted with ethyl acetate. The combined extracts were dried over magnesium sulfate and solvent removed under reduced pressure to afford title compound as a white solid, 2.0 g.

¹H NMR (DMSO-d6): δ 1.15 (t, J=7.4 Hz, 6H), 3.10 (q, J=7.4 Hz, 4H), 7.20 (m, 4H), 7.45 (m, 4H), 9.85 (br s, 2H).

### Preparation 7b: [3-(4-ethanesulfonylaminophenylsulfanyl)-6-fluoro-2-methylindolizin-1-yl]acetic acid ethyl ester

The title compound was prepared by the method of Preparation 2c using (6-fluoro-2-methylindolizin-1-yl)acetic acid ethyl ester and ethanesulfonic acid [4-(4-ethanesulfonylaminophenyldisulfanyl)phenyl]amide.

¹H NMR (CDCl₃): δ 1.25 (t, J=7.2 Hz, 3H), 1.35 (t, J=7.2 Hz, 3H), 2.35 (s, 3H), 3.05 (q, J=7.2 Hz, 2H), 3.75 (s, 2H), 4.15 (q, J=7.2 Hz, 2H), 6.25 (s, 1H), 6.75 (m, 1H), 6.80 (d, J=8.7 Hz, 2H), 7.05 (d, J=8.7 Hz, 2H), 7.40 (dd, J=5.1, 9.8 Hz, 1H), 8.1 (dd, J=2.1, 5.1 Hz, 1H).

### Preparation 7c: [3-(4-ethanesulfonylaminobenzenesulfonyl)-6-fluoro-2-methylindolizin-1-yl]acetic acid methyl ester

A mixture of [3-(4-ethanesulfonylaminophenylsulfanyl)-6-fluoro-2-methylindolizin-1-yl]acetic acid ethyl ester (0.030 g), 3-chloroperoxybenzoic acid (0.090 g), potassium carbonate (0.092 g) and methanol was stirred at room temperature for 3 hours. The mixture was concentrated under reduced pressure, diluted with water and extracted with dichloromethane. The combined extracts were washed with saturated aqueous sodium chloride solution, dried over magnesium sulfate and the solvent removed under reduced pressure to afford title compound.

MS: ESI (+ve) (Method B): 469 (M+H)⁺, Retention time 3.3 min.

### Preparation 7d: ethanesulfonylaminobenzenesulfonyl)-6-fluoro-2-methylindolizin-1-yl]acetic acid

The title compound was prepared by the method of Preparation 1d using [3-(4-ethanesulfonylaminobenzenesulfonyl)-6-fluoro-2-methylindolizin-1-yl]acetic acid ethyl ester.

MS: ESI (+ve) (Method A): 455 (M+H)⁺, Retention time 9.1 min.

MS: ESI (+ve) (Method B): 455 (M+H)⁺, Retention time 3.1 min.

### Example 8

### [7-chloro-6-fluoro-3-(4-methanesulfonylphenylsulfanyl)-2-methylindolizin-1-yl]acetic acid

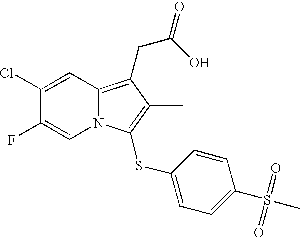

### Preparation 8a: [7-chloro-6-fluoro-3-(4-methanesulfonylphenylsulfanyl)-2-methylindolizin-1-yl]acetic acid ethyl ester

Sulfuryl chloride (0.40 mL) was added to a mixture of bis[4-(methylsulfonyl)phenyl]disulfide (1.6 g) and dichloromethane (40 mL) at 0° C. and the resulting mixture was stirred at 0° C. for 10 minutes and then at room temperature for 90 minutes. A solution of (6-fluoro-2-methylindolizin-1-yl)acetic acid ethyl ester (1.0 g) in dichloroethane (5.0 mL) was added and the resulting mixture stirred at room temperature overnight. The mixture was diluted with dichloromethane and saturated aqueous sodium hydrogen carbonate solution and the organic phase was washed with water and saturated aqueous sodium chloride solution and then dried over magnesium sulfate. The solvent was removed under reduced pressure and the residue purified by column chromatography on silica gel, eluting with a mixture of cyclohexane and ethyl acetate (9:1 to 85:15 by volume) to afford title compound.

MS: ESI (+ve) (Method B): 456 (M+H)⁺, Retention time 4.3 min.

### Preparation 8b: [7-chloro-6-fluoro-3-(4-methanesulfonylphenylsulfanyl)-2-methylindolizin-1-yl]acetic acid

The title compound was prepared by the method of Preparation 1d using (7-chloro-6-fluoro-3-(4-methanesulfonylphenylsulfanyl)-2-methylindolizin-1-yl]acetic acid ethyl ester.

¹H NMR (CDCl₃): δ 2.30 (s, 3H), 3.00 (s, 3H), 3.75 (s, 2H), 6.90 (d, J=8.6 Hz, 2H), 7.45 (d, J=7.0 Hz, 1H), 7.70 (d, J=8.6 Hz, 2H), 8.05 (d, J=4.8 Hz, 1H).

MS: ESI (+ve) (Method A): 428 (M+H)⁺, Retention time 11.8 min.

MS: ESI (+ve) (Method B): 428 (M+H)⁺, Retention time 3.6 min.

### Example 9

### [3-(2-chloro-4-methanesulfonylphenylsulfanyl)-7-cyano-2-methylindolizin-1-yl]acetic acid

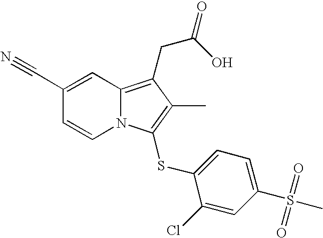

### Preparation 9a: 2-chloro-1-fluoro-4-methanesulfonylbenzene

A solution of sodium nitrite (5.7 g) in water (15 mL) was added dropwise over a period 15 minutes to a mixture of 2-fluoro-5-methanesulfonylphenylamine (15 g), concentrated hydrochloric acid (30 mL) and water (90 mL) at 0-10° C. The solution was added to a mixture of copper chloride(l) (11.8 g) and concentrated hydrochloric acid (30 mL) at 0° C. and the resulting mixture heated at 40° C. for 10 minutes. The mixture was cooled to room temperature, filtered and the filtrate extracted with dichloromethane. The combined extracts were dried over magnesium sulfate and the solvent removed under reduced pressure. Purification of the residue by column chromatography on silica gel, eluting with a mixture of toluene, dichloromethane and ethyl acetate (2:1:0 to 0:1:0 to 0:50:1 by volume) gave title compound as a white solid, 3.7 g.

¹H NMR (CDCl₃): δ 3.05 (s, 3H), 7.35 (t, J=8.4 Hz, 1H), 7.85 (m, 1H), 8.05 (dd, J=2.2, 6.6 Hz, 1H).

### Preparation 9b: bis(2-chloro-4-methanesulfonylbenzene)disulfide

A mixture of 2-chloro-1-fluoro-4-methanesulfonylbenzene (1.0 g), sodium hydrogensulfide (3.6 g) and 1-methylpyrrolidin-2-one (5.0 mL) was stirred at 80° C. for 90 minutes and then at room temperature for 4 hours. The mixture was diluted with water (40 mL) and acidified by the addition of concentrated hydrochloric acid. The resulting precipitate was collected by filtration, washed with water and ethanol and then crystallised from toluene to give title compound as a white solid, 0.61 g.

¹H NMR (DMSO-d6): δ 3.30 (s, 6H), 7.80 (d, J=8.3 Hz, 2H), 7.90 (dd, J=1.5, 8.3 Hz, 2H), 8.10 (br s, 2H).

MS: ESI (+ve) (Method B): 443 (M+H)⁺, Retention time 3.8 min.

### Preparation 9c: [3-(2-chloro-4-methanesulfonylphenylsulfanyl)-7-cyano-2-methylindolizin-1-yl]acetic acid ethyl ester

The title compound was prepared by the method of Preparation 2c using (7-cyano-2-methylindolizin-1-yl)acetic acid ethyl ester and bis(2-chloro-4-methanesulfonylbenzene)disulfide.

MS: ESI (+ve) (Method B): 463 (M+H)⁺, Retention time 3.7 min.

### Preparation 9d: [3-(2-chloro-4-methanesulfonylphenylsulfanyl)-7-cyano-2-methylindolizin-1-yl]acetic acid

A mixture of [3-(2-chloro-4-methanesulfonylphenylsulfanyl)-7-cyano-2-methylindolizin-1-yl]acetic acid ethyl ester (0.027 g), 5.0 M aqueous lithium hydroxide solution (0.25 mL), water (0.5 mL) and ethanol (5.0 mL) was stirred at room temperature for 1 hour. The mixture was acidified by the addition of glacial acetic acid, concentrated under reduced pressure and the residue partition between water (5.0 mL) and ethyl acetate (10 mL). The organic phase was dried over magnesium sulfate, concentrated under reduced pressure and the residue purified by preparative reverse-phase HPLC using a gradient over 30 minutes of acetonitrile in water (45% to 95% of organic modifier) to afford title compound, 0.019 g.

¹H NMR (DMSO-d6): δ 2.20 (s, 3H), 3.15 (s, 3H), 3.80 (s, 2H), 6.15 (d, J=8.6 Hz, 1H), 6.85 (dd, J=1.6, 7.1 Hz, 1H), 7.55 (dd, J =1.9, 8.4 Hz, 1H), 8.00 (d, J=1.9 Hz, 1H), 8.20 (dd, J=0.9, 7.1 Hz, 1H), 8.35 (dd, J=0.9, 1.6 Hz, 1H).

MS: ESI (+ve) (Method A): 435 (M+H)⁺, Retention time 10.7 min.

### Example 10

### [6-cyano-3-(4-methanesulfonylbenzyl)-2-methylindolizin-1-yl]acetic acid

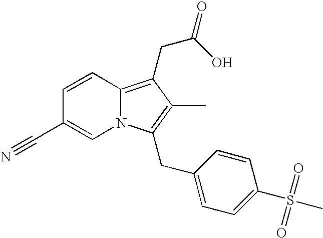

### Preparation 10a: 3-(5-cyanopyridin-2-yl)propionic acid ethyl ester

A solution of 3-ethoxy-3-oxopropylzinc bromide in tetrahydrofuran (0.5 M, 60 mL) was added dropwise over a period of 45 minutes to a mixture of 6-bromonicotinonitrile (5.0 g), tetrakis(triphenylphosphine)palladium(0), (0.69 g) and tetrahydrofuran (20 mL) at room temperature and the resulting mixture was stirred at room temperature for 6 hours. The mixture was diluted with water (150 mL), basified by the addition of saturated aqueous sodium hydrogen carbonate solution and extracted with methyl tert-butyl ether. The combined extracts were dried over magnesium sulfate and the solvent removed under reduced pressure. Purification of the residue by column chromatography on silica gel, eluting with a mixture of pentane, dichloromethane and ethyl acetate (2:1:0, 0:1:0 and 0:5:1 by volume) gave title compound as a pale yellow oil, 4.1 9.

¹H NMR (CDCl₃): δ 1.25 (m, 3H), 2.85 (t, J=7.2 Hz, 2H), 3.2 (t, J=7.2 Hz, 2H), 4.15 (m, 2H), 7.35 (d, J=8.0 Hz, 1H), 7.85 (m, 1H), 8.80 (s, 1H).

MS: ESI (+ve) (Method B): 205 (M+H)⁺, Retention time 2.7 min.

### Preparation 10b: (6-cyano-2-methylindolizin-1-yl)acetic acid ethyl ester

A mixture of 3-(5-cyanopyridin-2-yl)propionic acid ethyl ester (3.2 g), 1-bromopropan-2-one (5.3 g mL), sodium hydrogen carbonate (3.9 g) and acetonitrile (40 mL) was heated at reflux for 41 hours. The mixture was cooled to room temperature, diluted with water and extracted with ethyl acetate. The combined extracts were dried over magnesium sulfate and the solvent removed under reduced pressure. Purification of the residue by column chromatography on silica gel, eluting with a mixture of dichloromethane and pentane (1:4 to 1:1 by volume) gave title compound as a yellow solid, 0.51 g.

¹H NMR (CDCl₃): δ 1.25 (t, J=7.1 Hz, 3H), 2.30 (s, 3H), 3.65 (s, 2H), 4.10 (q, J=7.1 Hz, 2H), 6.65 (dd, J=1.4, 9.3 Hz, 1H), 7.20 (m, 1H), 7.35 (dd, J=0.9, 9.3 Hz, 1H), 8.20 (m, 1H).

MS: ESI (+ve) (Method B): 443 (M+H)⁺, Retention time 3.5 min.

### Preparation 10c: [6-cyano-3-(4-methanesulfonylbenzyl)-2-methylindolizin-1-yl]acetic acid ethyl ester

A mixture of (6-cyano-2-methylindolizin-1-yl)acetic acid ethyl ester (0.50 g), 4-methanesulfonylbenzaldehyde (0.38 g) and 1,2-dichloroethane (10 mL) at 0° C. was treated dropwise with triethylsilane (1.6 mL) followed by trifluoroacetic acid (0.46 mL) and the resulting mixture was stirred at 0° C. for 10 minutes and then at room temperature overnight. The mixture was diluted with 1,2-dichloroethane (10 mL), washed with saturated aqueous sodium hydrogen carbonate solution and dried over magnesium sulfate. The solvent was removed under reduced pressure and the residue purified by column chromatography on silica gel, eluting with a mixture of cyclohexane and ethyl acetate (1:0 to 0:1 by volume) to afford title compound as a biege solid, 0.43 g.

MS: ESI (+ve) (Method B): 411 (M+H)⁺, Retention time 3.3 min.

### Preparation 10d: [6-cyano-3-(4-methanesulfonylbenzyl)-2-methylindolizin-1-yl]acetic acid

The title compound was prepared by the method of Preparation 1d using [6-cyano-3-(4-methanesulfonylbenzyl)-2-methylindolizin-1-yl]acetic acid ethyl ester.

¹H NMR (DMSO-d6): δ 2.30 (s, 3H), 3.00 (s, 3H), 3.75 (s, 2H), 4.30 (s, 2H), 6.70 (dd, J=1.0, 9.3 Hz, 1H), 7.15 (d, J=8.5 Hz, 2H), 7.35 (dd, J=1.0, 9.3 Hz, 1H), 7.85 (m, 3H).

MS: ESI (+ve) (Method A): 383 (M+H)⁺, Retention time 8.8 min.

### Example 11

### [3-(4-chlorobenzyl)-7-cyano-2-methylindolizin-1-yl]acetic acid

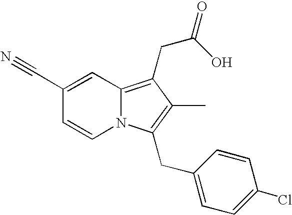

### Preparation 11a: 3-(4-cyanopyridin-2-yl)propionic acid ethyl ester

A solution of 3-ethoxy-3-oxopropylzinc bromide in tetrahydrofuran (0.5 M, 8.0 mL) was added dropwise to a mixture of 2-chloroisonicotinonitrile (0.50 g), tetrakis(triphenylphosphine)palladium(0) (0.21 g) and toluene (8.0 mL) at room temperature and the resulting mixture was stirred at 100° C. for 18 hours. The mixture was cooled to room temperature, diluted with saturated aqueous sodium hydrogen carbonate solution and extracted with ethyl acetate. The combined extracts were dried over sodium sulfate and the solvent removed under reduced pressure. Purification of the residue by column chromatography on silica gel, eluting with a mixture of cyclohexane and ethyl acetate (9:1 by volume) gave title compound, 0.44 g.

MS: ESI (+ve) (Method B): 205 (M+H)⁺, Retention time 2.8 min.

### Preparation 14b: (7-cyano-2-methylindolizin-1-yl)acetic acid ethyl ester

A mixture of 3-(4-cyanopyridin-2-yl)propionic acid ethyl ester (0.78 g), 1-bromopropan-2-one (1.3 g), sodium hydrogen carbonate (0.96 g) and acetonitrile (6.0 mL) was sealed in a flask and heated at 110° C. for 18 hours. The mixture was cooled to room temperature and the solvent removed under reduced pressure. Purification of the residue by column chromatography on silica gel, eluting with a mixture of cyclohexane and ethyl acetate (19:1 by volume) gave title compound, 0.29 g.

MS: ESI (+ve) (Method B): 243 (M+H)⁺, Retention time 3.6 min.

### Preparation 11c: [3-(4-chlorobenzyl)-7-cyano-2-methylindolizin-1-yl]acetic acid ethyl ester

The title compound was prepared by the method of Preparation 10c using (7-cyano-2-methylindolizin-1-yl)acetic acid ethyl ester and 4-chlorobenzaldehyde.

MS: ESI (+ve) (Method B): 367 (M+H)⁺, Retention time 4.3 min.

### Preparation 11d: [3-(4-chlorobenzyl)-7-cyano-2-methylindolizin-1-yl]acetic acid

The title compound was prepared by the method of Preparation 1d using [3-(4-chlorobenzyl)-7-cyano-2-methylindolizin-1-yl]acetic acid ethyl ester

¹H NMR (CDCl₃): δ 2.30 (s, 3H), 3.00 (s, 3H), 3.75 (s, 2H), 4.30 (s, 2H), 6.70 (dd, J=1.3, 9.3 Hz, 1H), 7.15 (d, J=8.5 Hz, 2H), 7.35 (dd, J=0.8, 9.3 Hz, 1H), 7.80 (m, 3H)

MS: ESI (+ve) (Method A): 339 (M+H)⁺, Retention time 11.2 min.

### Example 12

### [6-cyano-3-(6-fluoroquinolin-2-ylmethyl)-2-methylindolizin-1-yl]acetic acid

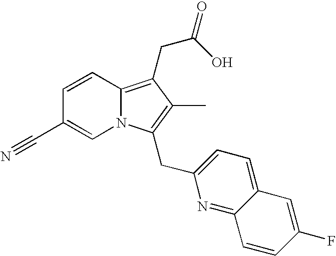

### Preparation 12a: [6-cyano-3-(6-fluoroquinolin-2-ylmethyl)-2-methylindolizin-1-yl]acetic acid ethyl ester

The title compound was prepared by the method of Preparation 10c using 6-cyano-2-methylindolizin-1-yl)acetic acid ethyl ester and 6-fluoroquinoline-2-carbaldehyde.

MS: ESI (+ve) (Method B): 402 (M+H)⁺, Retention time 4.0 min.

### Preparation 12b: [6-cyano-3-(6-fluoroquinolin-2-ylmethyl)-2-methylindolizin-1-yl]acetic acid

The title compound was prepared by the method of Preparation 1d using [6-cyano-3-(6-fluoroquinolin-2-ylmethyl)-2-methylindolizin-1-yl]acetic acid ethyl ester

¹H NMR (DMSO-d6): δ 2.25 (s, 3H), 3.80 (s, 2H), 4.65 (s, 2H), 6.80 (dd, J=1.4, 9.3 Hz, 1H), 7.35 (d, J=8.5 Hz, 1H), 7.55 (dd, J=1.0, 9.3 Hz, 1H), 7.65 (dt, J=3.1, 8.8 Hz, 1H), 7.75 (dd, J=2.9, 9.3 Hz, 1H), 7.95 (dd, J=5.4, 9.3 Hz, 1H), 8.30 (d, J=8.5 Hz, 1H), 9.05 (t, J=1.0 Hz, 1H).

MS: ESI (+ve) (Method A): 374 (M+H)⁺, Retention time 10.0 min.

### Example 13 and 14

### [6-fluoro-3-(4-methoxyphenylsulfanyl)-2-methylindolizin-1-yl]acetic acid and [7-chloro-6-fluoro-3-(4-methoxyphenylsulfanyl)-2-methylindolizin-1-yl]acetic acid

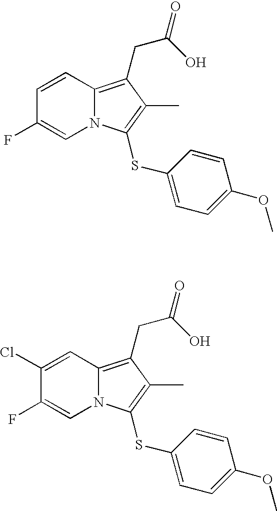

### Preparation 13a and 14a: [6-fluoro-3-(4-methoxyphenylsulfanyl)-2-methylindolizin-1-yl]acetic acid ethyl ester and [7-chloro-6-fluoro-3-(4-methoxyphenylsulfanyl)-2-methylindolizin-1-yl]acetic acid ethyl ester

The title compound was prepared by the method of Preparation 2c using (6-fluoro-2-methylindolizin-1-yl)acetic acid ethyl ester and bis(4-methoxyphenyl) disulfide.

MS: ESI (+ve) (Method B): Retention time 4.5 min.

### Preparation 13b and 14b: [6-fluoro-3-(4-methoxyphenylsulfanyl)-2-methylindolizin-1-yl]acetic acid and [7-chloro-6-fluoro-3-(4-methoxyphenylsulfanyl)-2-methylindolizin-1-yl]acetic acid

A mixture of [6-fluoro-3-(4-methoxyphenylsulfanyl)-2-methylindolizin-1-yl]acetic acid ethyl ester and [7-chloro-6-fluoro-3-(4-methoxyphenylsulfanyl)-2-methylindolizin-1-yl]acetic acid ethyl ester (0.35 g) and methanol (8.0 mL) was treated with 1.0 M aqueous sodium hydroxide solution (2.5 mL) and the resulting mixture was stirred at room temperature for 3 hours. The mixture was concentrated under reduced pressure, diluted with water and acidified by the addition of glacial acetic acid. The mixture was extracted with dichloromethane and the combined extracts were washed with water and dried over magnesium sulfate. The solvent was removed under reduced pressure and the residue purified by preparative reverse-phase HPLC using a gradient of acetonitrile in water (55% to 80% of organic modifier) to afford [6-fluoro-3-(4-methoxyphenylsulfanyl)-2-methylindolizin-1-yl]acetic acid as a pale green solid, 0.20 g and [7-chloro-6-fluoro-3-(4-methoxyphenylsulfanyl)-2-methylindolizin-1-yl]acetic acid as a pale yellow solid, 0.006 g.

[6-fluoro-3-(4-methoxyphenylsulfanyl)-2-methylindolizin-1-yl]acetic acid

¹H NMR (CDCl₃): δ 2.35 (s, 3H), 3.70 (s, 3H), 3.75 (s, 2H), 6.70-6.75 (m, 3H), 6.85 (d, J=8.9 Hz, 2H), 7.30 (dd, J=5.2, 9.7 Hz, 1H), 8.10 (m, 1H).

MS: ESI (+ve) (Method A): 346 (M+H)⁺, Retention time 11.7 min.

[7-chloro-6-fluoro-3-(4-methoxyphenylsulfanyl)-2-methylindolizin-1-yl]acetic acid

¹H NMR (CDCl₃): δ 2.35 (s, 3H), 3.70 (s, 3H), 3.75 (s, 2H), 6.70 (d, J=8.8 Hz, 2H), 6.85 (d, J=8.8 Hz, 2H), 7.40 (d, J=7.1 Hz, 1H), 8.15 (d, J=5.1 Hz, 1H).

MS: ESI (+ve) (Method A): 380 (M+H)⁺, Retention time 12.5 min.

### Example 15

### [3-(4-bromophenylsulfanyl)-6-fluoro-2-methylindolizin-1-yl]acetic acid

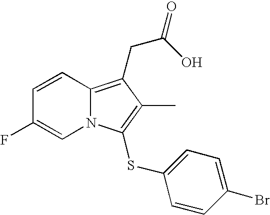

### Preparation 15a: [3-(4-bromophenylsulfanyl)-6-fluoro-2-methylindolizin-1-yl]acetic acid ethyl ester

The title compound was prepared by the method of Preparation 2c using (6-fluoro-2-methylindolizin-1-yl)acetic acid ethyl ester and 4-bromobenzenethiol.

MS: ESI (+ve) (Method B): Retention time 4.9 min.

### Preparation 15b: [3-(4-bromophenylsulfanyl)-6-fluoro-2-methylindolizin-1-yl]acetic acid

The title compound was prepared by the method of Preparation 13b and 14b using [3-(4-bromophenylsulfanyl)-6-fluoro-2-methylindolizin-1-yl]acetic acid ethyl ester.

¹H NMR CDCl₃): δ 2.30 (s, 3H), 3.75 (s, 2H), 6.65 (d, J=8.6 Hz, 2H), 6.75 (m, 1H), 7.25 (d, J=8.6 Hz, 2H), 7.35 (dd, J=5.3, 9.7 Hz, 1H), 8.00 (m, 1H).

MS: ESI (+ve) (Method A): 395 (M+H)⁺, Retention time 12.9 min.

### Example 16

### [3-(4-cyclopropylsulfamoylphenylsulfanyl)-6-fluoro-2-methylindolizin-1-yl]acetic acid

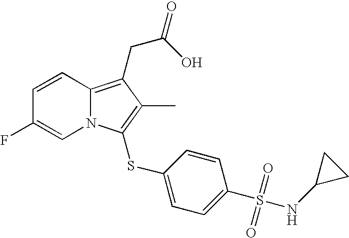

### Preparation 16a: 4,4′-dithiobisbenzenesulfonic acid, disodium salt

Prepared by the method of Smith et al. (J. Org. Chem., 1964, 29, 1484-1488).

### Preparation 16b: 4,4′-dithiobisbenzenesulfonyl chloride

A mixture of 4,4′-dithiobisbenzenesulfonic acid, disodium salt (6.2 g), phosphorus oxychloride (6.2 mL) and phosphorus pentachloride (3.1 g) was heated at reflux for 2 hours. The mixture was cooled to room temperature, diluted with dichloromethane and poured into ice. The organic phase was washed with saturated aqueous sodium hydrogen carbonate solution, dried over magnesium sulfate and the solvent removed under reduced pressure. The residue was triturated with cyclohexane to afford title compound as a light brown solid, 3.5 g.

¹H NMR (CDCl₃): δ 7.70 (m, 4H), 8.00 (m, 4H).

### Preparation 16c: bis[(cyclopropyl-4-sulfonyl)benzene]disulfide

A solution of 4,4′-dithiobisbenzenesulfonyl chloride (1.0 g) in dichloromethane (20 mL) was added dropwise to a mixture of cyclopropylamine (0.4 mL), triethylamine (1.6 mL) and dichloromethane (15 mL) at 0° C. and the resulting mixture was stirred at room temperature for 2 days. The mixture was concentrated under reduced pressure and the residue was purified by column chromatography on silica gel, eluting with a mixture of dichloromethane and methanol (1:0 to 99:1 by volume) to afford title compound as a pale yellow solid, 1.1 g.

MS: ESI (+ve) (Method B): 457 (M+H)⁺, Retention time 3.7 min.

### Preparation 16d: [3-(4-cyclopropylsulfamoylphenylsulfanyl)-6-fluoro-2-methylindolizin-1-yl]acetic acid ethyl ester

The title compound was prepared by the method of Preparation 2c using (6-fluoro-2-methylindolizin-1-yl)acetic acid ethyl ester and bis[(cyclopropyl-4-sulfonyl)benzene]disulfide.

MS: ESI (+ve) (Method B): 463 (M+H)⁺, Retention time 4.1 min.

### Preparation 16e: [3-(4-cyclopropylsulfamoylphenylsulfanyl)-6-fluoro-2-methylindolizin-1-yl]acetic acid

The title compound was prepared by the method of Preparation 1d using [3-(4-cyclopropylsulfamoylphenylsulfanyl)-6-fluoro-2-methylindolizin-1-yl]acetic acid ethyl ester.

¹H NMR (CDCl₃): δ 0.55 (m, 4H), 2.15 (m, 1H), 2.30 (s, 3H), 3.75 (s, 2H), 4.95 (br s, 1H), 6.80 (m, 1H), 6.90 (d, J=8.6 Hz, 2H), 7.35 (dd, J=5.1, 9.7 Hz, 1H), 7.65 (d, J=8.6 Hz, 2H), 8.00 (dd, J=1.9, 5.1 Hz, 1H).

MS: ESI (+ve) (Method A): 435 (M+H)⁺, Retention time 10.8 min.

Biological Results:

Compounds of the Examples above were, tested in the CRTH2 Radioligand Binding assay described above; the compounds had Ki values of less than 100 nM in the binding assay. For example, compounds of Examples 1, 2, 3, 4, 5, 6, 7, 8, 9, 10, 11, 12, 13, 14 and 16 had Ki values of 27, 0.2, 0.9, 29, 0.8, 57, 47, 0.4, 2.9, 1.7, 26, 7.2, 44, 23 and 0.6 nM, respectively. The compounds also generally had IC₅₀ values in the calcium flux functional assay of less than 100 nM. For example, compounds of Examples 2, 3, 5 and 16 had IC₅₀ values of 6.6, 11, 8.9 and 22 nM, respectively.

1. A compound selected from the group consisting of:
[3-(2,4-dichlorophenylsulfanyl)-6-fluoro-2-methylindolizin-1-yl]acetic acid,
[6-fluoro-3-(2-fluoro-4-methanesulfonylphenylsulfanyl)-2-methylindolizin-1-yl]acetic acid,
[6-fluoro-3-(4-methanesulfonyl-2-trifluoromethylphenylsulfanyl)-2-methylindolizin-1-yl]acetic acid,
(R)-2-[6-fluoro-3-(4-methanesulfonylphenylsulfanyl)-2-methylindolizin-1-yl]propionic acid,
(S)-2-[6-fluoro-3-(4-methanesulfonylphenylsulfanyl)-2-methylindolizin-1-yl]propionic acid,
[3-(4-ethanesulfonylphenylsulfanyl)-6-fluoro-2-methylindolizin-1-yl]acetic acid,
ethanesulfonylaminobenzenesulfonyl)-6-fluoro-2-methylindolizin-1-yl]acetic acid,
[7-chloro-6-fluoro-3-(4-methanesulfonylphenylsulfanyl)-2-methylindolizin-1-yl]acetic acid,
[3-(2-chloro-4-methanesulfonylphenylsulfanyl)-7-cyano-2-methylindolizin-1-yl]acetic acid,
[6-cyano-3-(4-methanesulfonylbenzyl)-2-methylindolizin-1-yl]acetic acid,
[3-(4-chlorobenzyl)-7-cyano-2-methylindolizin-1-yl]acetic acid,
[6-cyano-3-(6-fluoroquinolin-2-ylmethyl)-2-methylindolizin-1-yl]acetic acid,
[6-fluoro-3-(4-methoxyphenylsulfanyl)-2-methylindolizin-1-yl]acetic acid,
[7-chloro-6-fluoro-3-(4-methoxyphenylsulfanyl)-2-methylindolizin-1-yl]acetic acid,
[3-(4-bromophenylsulfanyl)-6-fluoro-2-methylindolizin-1-yl]acetic acid,
[3-(4-cyclopropylsulfamoylphenylsulfanyl)-6-fluoro-2-methylindolizin-1-yl]acetic acid, and salts, N-oxides, hydrates and solvates thereof.

2. (canceled)

3. A pharmaceutical composition comprising a compound as claimed in claim 1 and a pharmaceutically acceptable carrier.

4-5. (canceled)

6. A method of treatment of asthma, chronic obstructive pulmonary disease, rhinitis, allergic airway syndrome, or allergic rhinobronchitis, comprising administering to a patient suffering such disease an effective amount of a compound as claimed in claim 1.

7. A method of treatment of atopic or non-atopic dermatitis, Crohn's disease, ulcerative colitis, or irritable bowel disease, comprising administering to a patient suffering such disease an effective amount of a compound as claimed in claim 1.

8. (canceled)
